(19) United States
(12) Patent Application Publication
(10) Pub. No.: US 2010/0113899 A1
(43) Pub. Date: May 6, 2010

(54) Alignment System for Optically Sampling a Hand

(76) Inventors: Mark Ries Robinson, Albuquerque, NM (US); Andrea Magee, Albuquerque, NM (US); Michael U. Haass, Albuquerque, NM (US); Russell E. Abbink, Sandia Park, NM (US); William Radigan, Albuquerque, NM (US); V. Gerald Grafe, Corrales, NM (US)

Correspondence Address: V. Gerald Grafe, esq., P.O. Box 2689, Corrales, NM, 87048, US

(21) Appl. No.: 12/614,056

(22) Filed: Nov. 6, 2009

Related U.S. Application Data
(60) Provisional application No. 61/111,815, filed on Nov. 6, 2008.
(60) Provisional application No. 61/122,178, filed on Dec. 12, 2008.

(51) Int. Cl. A61B 5/1455

(52) U.S. Cl. 600/310

(57) ABSTRACT

This invention relates to measurements of material properties by determination of the response of a sample to incident radiation, and more specifically to the measurement of analytes such as glucose or alcohol in human tissue. Some example embodiments of the present invention provide an optical sampling apparatus including an optical subsystem, having a receiver for receiving light expressed from tissue; and an alignment subsystem, adapted to urge a portion of a hand placed in operative relationship with the alignment subsystem into a defined configuration relative to the receiver. In some such example embodiments, the alignment subsystem includes a substrate having a plurality of projections therefrom disposed in a pattern such that the projections urge a portion of a hand placed on the substrate to a defined configuration relative to the receiver. In some such example embodiments, the alignment subsystem includes a substrate having a surface defining a volume approximating the shape of a portion of a hand such that the volume urges a portion of a hand placed therein to a defined configuration relative to the receiver. In some such example embodiments, the alignment subsystem includes a surface having a generally U-shaped cross-section and extending for a distance, wherein the surface is mounted relative to the sampling means such that a finger placed in the U-shaped cross-section of the surface is urged to a defined configuration relative to the receiver. In some such sample embodiments, the alignment subsystem is customized to portion of the hand of a specific individual.

### CROSS-REFERENCE TO RELATED APPLICATIONS

This application claims priority to U.S. provisional application 61/111,815, filed Nov. 6, 2008, and to U.S. provisional application 61/122,178, filed Dec. 12, 2008, each of which is incorporated herein by reference. This application is related to U.S. application Ser. No. 12/239,601, filed Sep. 26, 2008, which is incorporated herein by reference.

### BACKGROUND OF THE INVENTION

This invention relates to measurements of material properties by determination of the response of a sample to incident radiation, and more specifically to the measurement of analytes such as glucose or alcohol in human tissue.

Noninvasive glucose monitoring has been a long-standing objective for many development groups. Several of these groups have sought to use near infrared spectroscopy as the measurement modality. To date, none of these groups has demonstrated a system that generates noninvasive glucose measurements adequate to satisfy both the U.S. Food and Drug Administration (“FDA”) and the physician community. Spectroscopic noise introduced by the tissue media is a principal reason for these failures. Tissue noise can include any source of spectroscopic variation that interferes with or hampers accuracy of the analyte measurement. Changes in the optical properties of tissue can contribute to tissue noise. The measurement system itself can also introduce tissue noise, for example changes in the system can make the properties of the tissue appear different. Tissue noise has been well recognized in the published literature, and is variously described as physiological variation, changes in scattering, changes in refractive index, changes in pathlength, changes in water displacement, temperature changes, collagen changes, and changes in the layer nature of tissue. See, e.g., Khalil, Omar: Noninvasive glucose measurement technologies: an update from 1999 to the dawn of the new millennium. Diabetes Technology & Therapeutics, Volume 6, number 5, 2004. Variations in the optical properties of tissue can limit the applicability of conventional spectroscopy to noninvasive measurement. Conventional absorption spectroscopy relies on the Beer-Lambert-Bouger relation between absorption, concentration, pathlength, and molar absorptivity. For the single wavelength, single component case:

I_{λ}=I_{λ,o}10^{-ε}^{_{λ}}^{lc}

a_{λ}=ε_{λ}lc

Where I_{λ,o} and I_{λ} are the incident and excident flux, is the molar absorptivity, c is the concentration of the species, and l is the pathlength through the medium. a is the absorption at wavelength (−log₁₀(I_{λ}/I_{λ,o})). These equations assume that photons either pass through the medium with pathlength l, or are absorbed by the molecular occupants.

Unfortunately, optical measurement of tissue does not match the assumptions required by Beer's law. Variations in tissue between individuals, variations in tissue between different locations or different times with the same individual, surface contaminants, interaction of the measurement system with the tissue, and many other real-world effects can prevent accurate optical measurements. There is a need for improvements in optical measurement methods and apparatuses that allow accurate measurements in real-world settings.

Noninvasive glucose measurement devices that sample forearm tissue have been proposed. See, e.g., U.S. Pat. No. 6,574,490; U.S. Pat. No. 6,865,408; U.S. Pat. No. 6,990,364; U.S. Pat. No. 7,133,710; each of which is incorporated herein by reference. The forearm can be a desirable site for tissue measurements for several reasons. As an example, systems can be devised that allow highly reproducible selection of sampling site, which can be important to help reduce measurement error arising from sampling of different tissue volumes. See, e.g., U.S. Pat. No. 7,206,623; U.S. Pat. No. 7,233,816; each of which is incorporated herein by reference. A forearm sampling site can pose difficulties, however, especially when comparing results to measurements obtained from blood from conventional “finger stick” meters. There is a need for tissue sampling methods and apparatuses that allow consistent sampling of substantially the same tissue volume, particularly when sampling tissue of the hand.

### SUMMARY OF THE INVENTION

Some example embodiments of the present invention provide an optical measurement apparatus comprising a sampling means for optically sampling tissue, and alignment means for accepting a portion of a hand and placing a portion of a hand in a defined configuration relative to the sampling means. “A portion of a hand” can mean all or part of a hand, for example, a single finger, several fingers, several fingers plus the heel, or the whole hand. A “configuration” of a portion of the hand refers to one or more characteristics of the hand than can be significant in optically sampling tissue, for example the physical placement of a portion of the hand, the rotation of the portion, the state of stretch of the skin or underlying tissue, the state of compression of the portion or the underlying tissue, the spacing or rotation of one portion of hand relative to another portion (e.g., the spread of several fingers).

Some of such example embodiments provide an alignment means having a substrate having a plurality of projections therefrom disposed in a pattern such that the projections urge a portion of a hand placed on the substrate to a defined configuration relative to the sampling means. Some of such example embodiments provide an alignment means having a surface defining a volume approximating the shape of a portion of a hand such that the volume urges a portion of a hand placed therein to a defined configuration relative to the sampling means. Some of such example embodiments provide an alignment means having a generally U-shaped cross-section and extending for a distance, wherein the surface is mounted relative to the sampling means such that a finger placed in the U-shaped cross-section of the surface is urged to a defined configuration relative to the sampling means. Some of such example embodiments provide an alignment means that is customized to a portion of the hand of a specific individual.

Some example embodiments of the present invention provide an optical sampling apparatus comprising an optical subsystem, having a receiver for receiving light expressed from tissue; and an alignment subsystem, adapted to urge a portion of a hand placed in operative relationship with the alignment subsystem into a defined configuration relative to the receiver. In some such example embodiments, the alignment subsystem comprises a substrate having a plurality of projections therefrom disposed in a pattern such that the projections urge a portion of a hand placed on the substrate to a defined configuration relative to the receiver. In some such example embodiments, the alignment subsystem comprises a substrate having a surface defining a volume approximating the shape of a portion of a hand such that the volume urges a portion of a hand placed therein to a defined configuration relative to the receiver. In some such example embodiments, the alignment subsystem comprises a surface having a generally U-shaped cross-section and extending for a distance, wherein the surface is mounted relative to the sampling means such that a finger placed in the U-shaped cross-section of the surface is urged to a defined configuration relative to the receiver. In some such sample embodiments, the alignment subsystem is customized to portion of the hand of a specific individual.

Some example embodiments of the present invention provide a method of optically sampling a portion of a hand of an individual, the method comprising (a) providing an alignment system adapted to place a portion of a hand of a subject in a defined configuration; (b) placing a portion of the hand of a subject in operative relationship with the alignment system; and (c) using an optically sampling apparatus to sample a portion of the hand corresponding to the portion that is placed in a known configuration relative to the optically sampling apparatus. In some such example embodiments, the alignment system comprises a surface having a plurality of projections therefrom disposed in a pattern such that the projections urge a portion of a hand placed on the substrate to a defined configuration, and wherein step b comprises placing the portion of the hand such that the hand engages the projections and assumes the defined configuration. In some such example embodiments, the alignment system comprises a substrate having a surface defining a volume approximating the shape of a portion of a hand such that the volume urges a portion of a hand placed therein to the defined configuration, and wherein step b comprises placing the portion of the hand such that the hand engages the volume and assumes the known configuration. In some such example embodiments, the alignment system comprises a surface having a generally U-shaped cross-section and extending for a distance, and wherein step b comprises placing a finger of the hand in the U-shaped cross-section. In some such example embodiments, the alignment system is customized to a portion of a hand of a specific individual.

### BRIEF DESCRIPTION OF THE DRAWINGS

FIG. 1 is a schematic illustration of an analyte measurement system according to the present invention.

FIG. 2(a,b,c,d) are schematic illustrations of example embodiments of the present invention.

FIG. 3(a,b,c,d) are schematic illustrations of an example embodiment of the present invention.

FIG. 4 is a schematic illustration of an example embodiment of the present invention.

FIG. 5 is a schematic illustration of an example embodiment of the present invention.

FIG. 6 is a schematic illustration of an example embodiment of the present invention.

### DESCRIPTION OF INVENTION

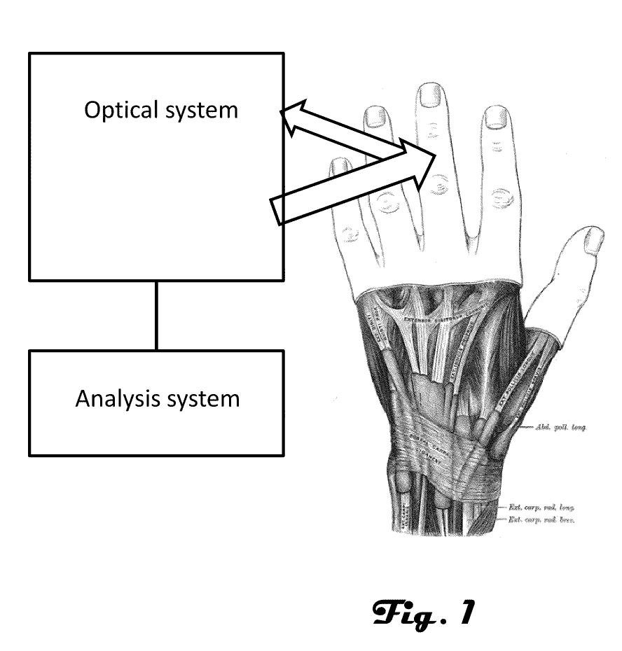

FIG. 1 is a schematic illustration of an analyte measurement system according to the present invention. An optical system communicates illumination electromagnetic energy to tissue to be sampled, and collects electromagnetic energy expressed from the tissue responsive to such illumination electromagnetic energy. An analysis system analyzes the collected light to determine properties of the tissue. In one example embodiment, the optical system comprises an illumination source and an illumination optical system adapted to transmit energy from the illumination source to the tissue, and a detection optical system to collect energy from the tissue and transmit it to a detector. The output of the detector is then analyzed by a programmed computer. Example optical systems that can be suitable for use with the present invention include those described in the patents and applications referenced elsewhere herein, and those described in patents and applications assigned to InLight Solutions and Sensys Medical. Some of those examples contemplate sampling on the forearm, and accordingly would be adapted to accommodate sampling on the hand as contemplated in the present invention. Embodiments of the present invention can accommodate sampling on any body part that can be reliably positioned according to any of the methods and apparatuses described herein, although the descriptions that follow generally assume sampling on a hand or finger for simplicity of illustration.

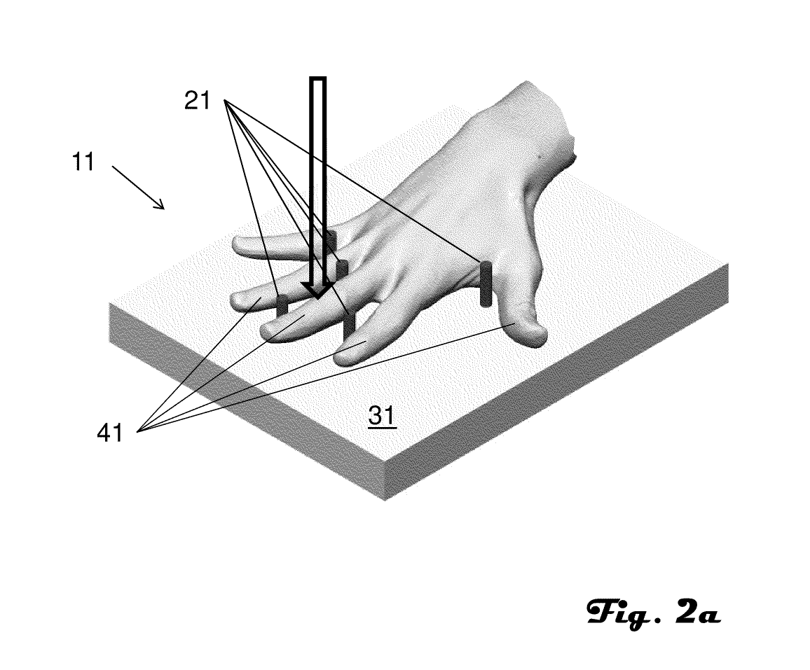

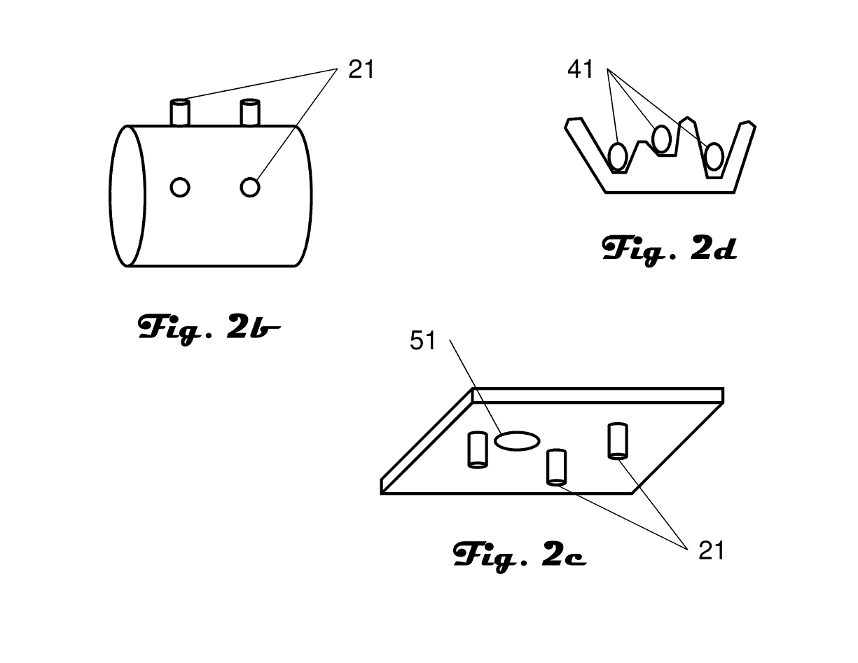

FIG. 2(a,b,c,d) are schematic illustrations of example embodiments of the present invention. In FIG. 2a, a hand sampling alignment device 11 interfaces with a measurement system (not shown) such as those described elsewhere herein. The hand sampling alignment device comprises a plurality of features disposed such that portions of the hand 41 contact each feature. In the figure, the features are shown as pins 21 disposed on and protruding from a flat plate 31, although a variety of different features can be used and they can be mounted relative to each in a variety of different ways. As examples, pins can protrude upward from a surface on which the hand rests, pins can protrude downward from a surface under which a hand is placed (e.g., FIG. 2c, with a window 51 for optical sampling), features such as pins or grooves can be disposed at various angles to each other, features can comprise walls or vanes or irregular-shaped objects mounted with each other to encourage a desired presentation of a portion of the hand to the sampler (e.g., FIG. 2d), features such as any of the preceding can be mounted with a surface, a grid, an expandable framework, a flat or curved surface (e.g., FIG. 2b), or with spacers placed between the features.

In the example embodiment shown in FIG. 2a, a first pin provides a reference position between the thumb and the index finger near the bases thereof. A second pin provides a reference position for the end of the index finger on the side facing the middle finger. A third pin provides a reference position for the end of the middle finger on the side facing the ring finger. A fourth pin provides a reference position between the middle and ring fingers, near the bases thereof. A fifth pin provides a reference position between the ring and pinky fingers near the bases thereof. A user can place the hand on the supporting surface and engage the five pins, which will position the hand, including the various tissue surfaces of the fingers, in a reproducible relationship to the pins. Such placement can facilitate sampling substantially the same tissue on separate insertions of the hand by an optical sampling system positioned relative to the pins. As an example, an optical sampling system can sample tissue on the middle finger at a consistent location thereon. The pins provide assurance that not only will the middle finger be at the expected location, but the associated tissue will have a similar orientation in three dimensions and compression or stretching condition with each insertion of the hand into the alignment apparatus. Other numbers and arrangements of pins (or other features) can be used to advantageously position other fingers or parts of the hand.

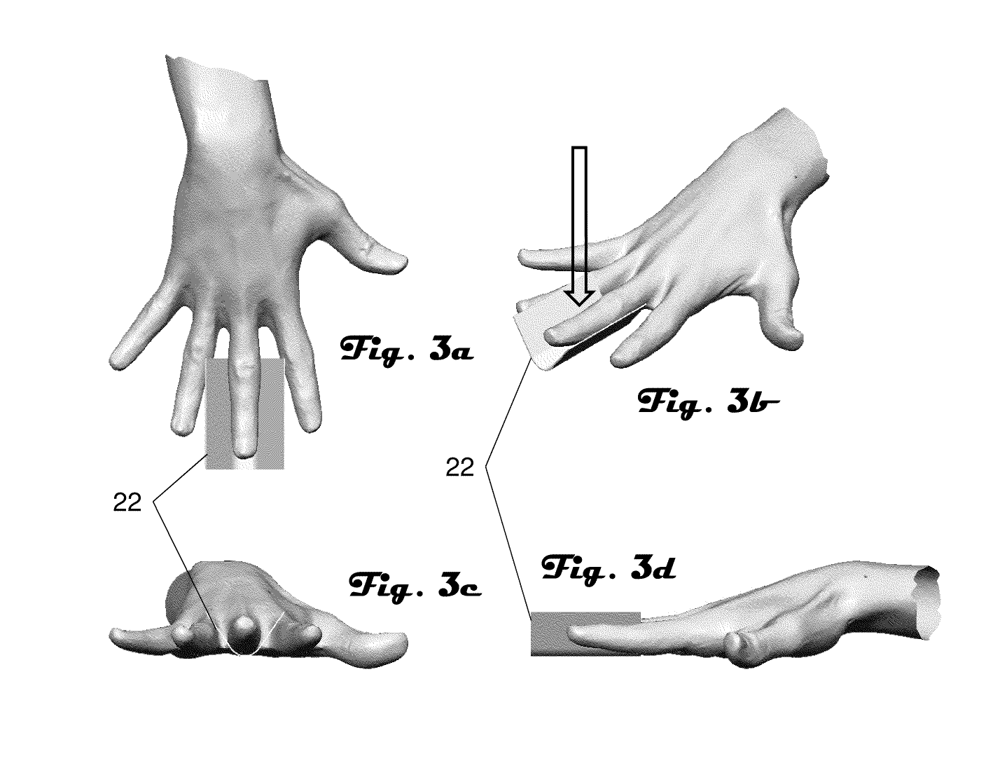

FIG. 3(a,b,c,d) are schematic illustrations of an example embodiment of the present invention. The example embodiment is shown from the top in FIG. 3a, from an upper perspective in FIG. 3b, from the side looking onto the end of the fingers of a hand placed in the embodiment in FIG. 3c, and from the side of the hand in FIG. 3d. A locating feature comprises a channel 22 shaped and sized to accommodate a finger, in the figure the middle finger although any finger can be used depending on the overall requirements of the measurement system and the preferred sampling site. As an example, a thin piece of bendable material such as metal, cardboard, or plastic can be bent into an appropriate shape and mounted relative to an optical sampling system (not shown). As another example, a suitable channel can be cut or machined from a suitable substrate material such as metal, plastic, or wood. As another example, a suitable locating feature can be molded from a material such as plastic with an appropriate channel formed as part of the molding process. As another example, a compliant material such as a film or other flexible or moldable material can be provided and the channel formed by the placement of the user's finger on the material. The central portion of the channel provides a consistently locatable centerline and base for the finger. The raised boundaries of the channel encourage the finger to rest in a consistent relation to the channel, and discourage interaction with or interference from other fingers. An optical sampling system can sample a volume located relative to the locating feature, and thereby consistently sample substantially the same portion of the hand. The channel can also facilitate heating or cooling of the tissue, if that is desired, by heating or cooling the channel material or by circulating heated or cooled air into, out of, or along the channel. Note that this embodiment, as well as other embodiments described herein, provide location advantages when sampling different hands by presenting a similar portion of the hand to the sampling system. When repeatedly sampling the same hand, moreover, the present invention can facilitate sampling the same tissue volume with a very high degree of reproducibility due to the repeatability of the mechanical interface of the hand with the channel.

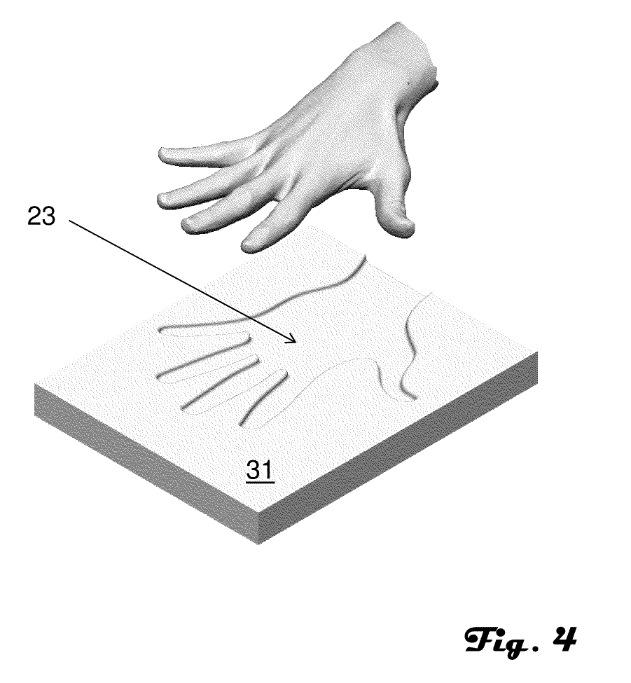

FIG. 4 is a schematic illustration of an example embodiment of the present invention. A supporting substrate 31 has formed therein or thereon a hand-receiving shape 23 that closely conforms a specific hand shape. The shape can be specific to an individual hand in some embodiments, or specific to a range of overall hand shapes in other embodiments. If the shape is formed specific to an individual hand, then the hand can be located relative to the substrate with very high precision and repeatability. The example in the figure shows a recess matching the shape of the entire hand; other example embodiments can have recesses matching only portions of the hand (e.g., just a subset of the fingers). The hand-receiving shape can comprise an outline or two dimensional projection of the hand or portion of the hand, and can also comprise varying depths to provide a hand-receiving shape that conforms to the hand in three dimensions. The example in the figure corresponds to placement of the palm side of the hand; other example embodiments can correspond to placement of the back side of the hand, or portions of the hand at various other angles, depending on the requirements of the overall measurement system and the desired tissue sampling site.

Suitable hand-receiving shapes can be fabricated using techniques such as plaster casting, lost wax casting, investment casting, computer scanning and production, hand-fitting and sculpting of a workpiece, forming an impression in a moldable substrate and subsequent curing of the substrate, or other techniques known to those skilled in the art. An optical sampling system can sample a volume located relative to the hand-receiving shape, and thereby consistently sample substantially the same portion of the hand. The hand-receiving shape can also facilitate heating or cooling of the tissue, if that is desired, by heating or cooling the substrate material or by circulating heated or cooled air into, out of, or along the hand-receiving shape. When repeatedly sampling the same hand, moreover, the present invention can facilitate sampling the same tissue volume with a very high degree of reproducibility due to the mechanical interface of the hand with the hand-receiving shape.

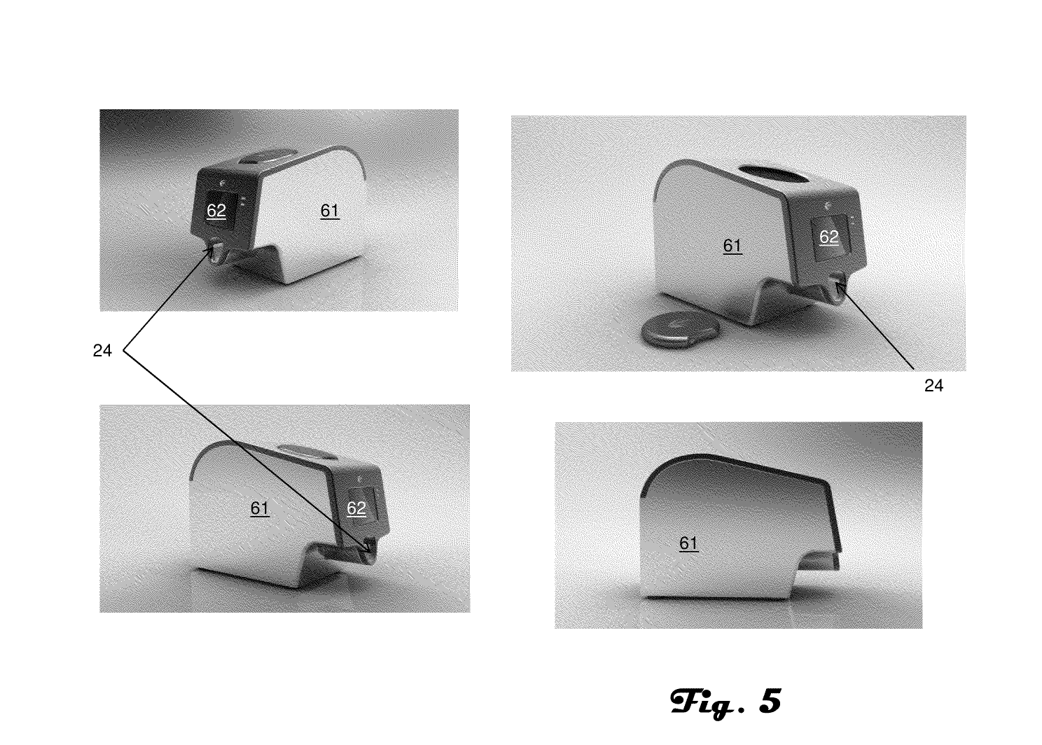

FIG. 5 is a schematic illustration of an example embodiment of the present invention. An optical sampling system is contained within the housing 61 shown in the figure. A display 62 capable of communicating instructions and results to a user is located on the front of the housing. A finger of the hand is presented to the optical sampling system by inserting the finger in the opening 24 below the display on the front of the housing. The opening is shaped such that a finger placed therein is automatically positioned in a defined relationship to the optical sampling system. The shape of the opening defines a channel with inwardly sloping sides and a closed bottom, such that a finger placed therein is urged to a defined position, e.g., centered, relative to the sides, and positioned at a height defined by the interaction of the finger with the sides and the bottom of the channel.

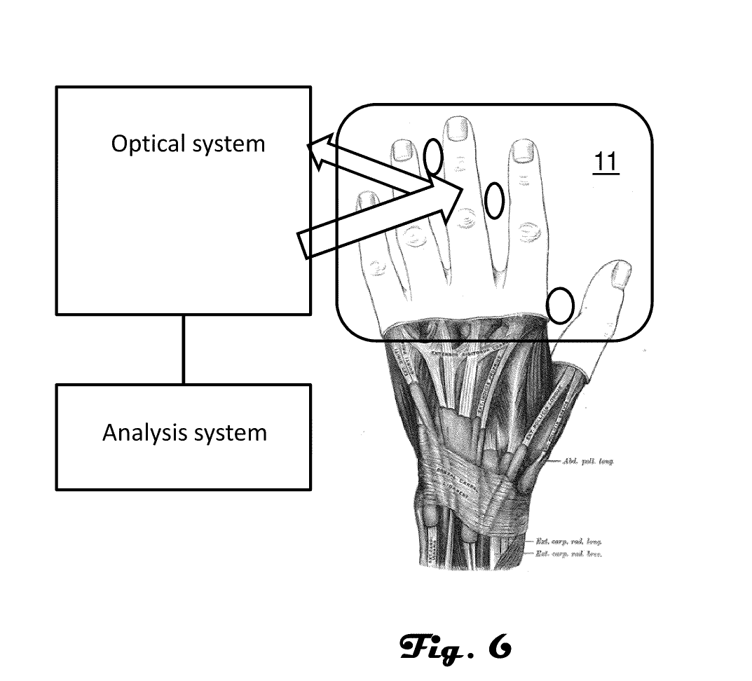

The present invention(s) also provides methods of determining tissue properties, such as the presence or concentration of analytes, including measurements of glucose concentration in tissue. An example embodiment of such a method comprises supplying an alignment apparatus such as those described or enabled herein, in operative relationship to an optical sampling system, as illustrated schematically in FIG. 6. A user presents a hand to the alignment system 11 and the optical system provides illumination energy and detects light expressed from the tissue responsive to the illumination energy. Illumination energy and detected light can comprise visible light, heat, infrared light, ultraviolet light, mid-infrared light, near-infrared light, other forms of energy or wavelengths of light, and any combination or subset thereof. An analysis system analyzes the collected light and determines the tissue property, e.g., the glucose concentration, for example using multivariate methods such as those described in the patents and applications incorporated herein. The user subsequently presents the hand to the alignment device, and the alignment device urges the hand to substantially the same relationship with the optical system as in the previous presentation, and the illumination, detection, and analysis steps repeated.

The present invention(s) also comprises methods of producing alignment systems such as those described or enabled herein. Examples of such methods include methods of producing hand-receiving shapes as described above; methods of mounting alignment features such as pins in relationship to each other such that a portion of a hand suitable for optical sample can be reliably positioned relative to the alignment features; and methods of producing channels by techniques such as those described above. The present invention also comprises methods for making tissue measurement systems, such as noninvasive glucose measurement systems, comprising making an alignment system according to the present invention, making an optical system suitable for use with the alignment system and suitable for determining interaction of the tissue with light sufficient to determine the tissue property, making an analysis system such as a multivariate analysis system, and integrating the aforementioned elements into a tissue measurement system.

The present invention(s) can also provide sensing capability combined with the above-described alignment capability. For example, contact or pressure sensors can detect when a hand is properly positioned in a channel or relative to alignment features or a hand-receiving shape. For example, optical or capacitive sensors can detect when a hand is properly positioned in a channel or relative to alignment features or a hand-receiving shape. Following insertion of the body part into the optical system using a given guidance mechanism, the system then detects if the body part is aligned correctly. If the body part is in an acceptable position the system initiates an optical scanning or measurement process. The scanning or measurement process continues until either the measurement has been completed or the system detects movement or misalignment of the body part. If misalignment is detected, then the system can notify the user and can provide prompting messages to re-align the body part. Upon realignment the system can automatically start the measurement process. In use the measurement process can be re-started or simply continued until enough measurements have been made.

The present invention has been described as an indwelling fiber optic probe. It will be understood that the above description is merely illustrative of the applications of the principles of the present invention, the scope of which is to be determined by the claims viewed in light of the specification. Other variants and modifications of the invention will be apparent to those of skill in the art.

We claim:

1. An optical measurement apparatus, comprising:
a. sampling means for optically sampling tissue;
b. alignment means for accepting a hand and placing a portion of the hand in a defined configuration relative to the sampling means.

2. An apparatus as in claim 1, wherein the alignment means comprises a substrate having a plurality of projections therefrom disposed in a pattern such that the projections urge a portion of a hand placed on the substrate to a defined configuration relative to the sampling means.

3. An apparatus as in claim 1, wherein the alignment means comprises a substrate having a surface defining a volume approximating the shape of a portion of a hand such that the volume urges a portion of a hand placed therein to a defined configuration relative to the sampling means.

4. An apparatus as in claim 1, wherein the alignment means comprises a surface having a generally U-shaped cross-section and extending for a distance, wherein the surface is mounted relative to the sampling means such that a finger placed in the U-shaped cross-section of the surface is urged to a defined configuration relative to the sampling means.

5. An apparatus as in claim 1, wherein the alignment means is customized to a portion of the hand of a specific individual.

6. An optical sampling apparatus, comprising:
a. an optical subsystem, having a receiver for receiving light expressed from tissue;
b. an alignment subsystem, adapted to urge a portion of a hand placed in operative relationship with the alignment subsystem into a defined configuration relative to the receiver.

7. An optical sampling apparatus as in claim 6, wherein the alignment subsystem comprises a substrate having a plurality of projections therefrom disposed in a pattern such that the projections urge a portion of a hand placed on the substrate to a defined configuration relative to the receiver.

8. An optical sampling apparatus as in claim 6, wherein the alignment subsystem comprises a substrate having a surface defining a volume approximating the shape of a portion of a hand such that the volume urges a portion of a hand placed therein to a defined configuration relative to the receiver.

9. An optical sampling apparatus as in claim 6, wherein the alignment subsystem comprises a surface having a generally U-shaped cross-section and extending for a distance, wherein the surface is mounted relative to the sampling means such that a finger placed in the U-shaped cross-section of the surface is urged to a defined configuration relative to the receiver.

10. An optical sampling apparatus as in claim 6, wherein the alignment subsystem is customized to a portion of the hand of a specific individual.

11. A method of optically sampling a portion of a hand of an individual, comprising:
a. providing an alignment system adapted to place a portion of a hand of a subject in a defined configuration;
b. placing a portion of the hand of a subject in operative relationship with the alignment system; and
c. using an optically sampling apparatus to sample a portion of the hand corresponding to the portion that is placed in a known configuration relative to the optically sampling apparatus.

12. A method as in claim 11, wherein the alignment system comprises a surface having a plurality of projections therefrom disposed in a pattern such that the projections urge a portion of a hand placed on the substrate to a defined configuration, and wherein step b comprises placing the portion of the hand such that the hand engages the projections and assumes the defined configuration.

13. A method as in claim 11, wherein the alignment system comprises a substrate having a surface defining a volume approximating the shape of a portion of a hand such that the volume urges a portion of a hand placed therein to the defined configuration, and wherein step b comprises placing the portion of the hand such that the hand engages the volume and assumes the known configuration.

14. A method as in claim 11, wherein the alignment system comprises a surface having a generally U-shaped cross-section and extending for a distance, and wherein step b comprises placing a finger of the hand in the U-shaped cross-section.

15. A method as in claim 11, wherein the alignment system is customized to a portion of the hand of a specific individual.
